(19) United States
(12) Patent Application Publication
(10) Pub. No.: US 2010/0319694 A1
(43) Pub. Date: Dec. 23, 2010

(54) Delivery of powdered drug via inhalation

(76) Inventors: Robert Owen Cook, Hillsborough, NJ (US); Veronica S. De Guzman, Palo Alto, CA (US); Thomas A. Armer, Cupertino, CA (US); Timothy S. Nelson, Los Gatos, CA (US)

Correspondence Address: Owen J. Bates, Suite 200, 2400 Bayshore Parkway, Mountain View, CA, 94043, US

(21) Appl. No.: 12/661,239

(22) Filed: Mar. 11, 2010

Related U.S. Application Data
(63) Continuation of application No. 12/460,688, filed on Jul. 22, 2009, now abandoned.
(60) Provisional application No. 61/135,832, filed on Jul. 23, 2008.

(51) Int. Cl. A61M 15/00

(52) U.S. Cl. 128/203.15

(57) ABSTRACT

Described herein are hand-held inhalation devices for use with tidal breathing that deliver powdered active agents to the respiratory system. The inhalation devices deliver a unit dose of the powdered active agent over a series of inhalations during tidal breathing. Methods for using the inhalation devices and kits for inhalation delivery of various active agents are also described.

### CROSS-REFERENCE TO RELATED APPLICATIONS

This application is a continuation of U.S. patent application Ser. No. 12/460,688, filed on 22 Jul. 2009, which claims the benefit of U.S. Provisional Application No. 61/135,832, filed 23 Jul. 2008, entitled “The Delivery of Powdered Drug via Inhalation” which is hereby incorporated by reference in its entirety.

### FIELD

The methods and devices described herein are in the field of inhalation drug delivery. Specifically, they relate to an inhaler that delivers active agents in dry powder form to the respiratory tract in response to repetitive, non-forced inhalations. Methods and kits for delivering dry powder medicament to treat various allergic and inflammatory conditions, as well as various pulmonary conditions are also described.

### BACKGROUND

Aerosols have been used for delivering active pharmaceutical agents to the lungs since the mid 1950s. For example, in the treatment of asthma, inhalers are commonly used for delivering aerosolized bronchodilators and corticosteroids. Three types of inhalers are in common use: aqueous nebulizers (nebulizers), metered dose inhalers (MDIs) and dry powder inhalers (DPIs). All have as their object the delivery of an active agent to the respiratory tract, either in the form of aerosol mists comprised of droplets containing suspended or dissolved medicaments or aerosol dusts with the medicament in the form of a finely divided powder.

In the MDIs, the active agent is usually provided by the pharmaceutical manufacturer in a pressurized aerosol canister, with the medication being suspended or dissolved in a liquid propellant such as a chlorofluorocarbon (CFC) or hydrofluororalkane (HFA). The canister includes a metering valve having a hollow discharge stem which can be depressed inward into the canister to discharge a metered volume of propellant-medication mixture in the form of an aerosol comprising fine droplets of propellant in which particles of the active agent are suspended or dissolved.

A typical MDI for use with such a canister includes a housing having an actuator and nozzle. The canister is inserted into the housing with the hollow discharge stem of the canister being received in a bore in the actuator. Depressing the closed end of the canister causes the stem to be pushed inward into the canister so that a metered volume of active agent is discharged through the nozzle. The housing further defines a flow path in fluid communication with the nozzle, the flow path having an outlet at a mouthpiece portion of the housing, such that the aerosolized active agent may be inhaled after it exits the mouthpiece. The patient typically inserts the mouthpiece into the mouth with the lips closed around the mouthpiece. The patient then depresses the canister to discharge the active agent, and simultaneously inhales, entraining the active agent aerosol into the inhaled breath.

DPIs contain the active agent in a variety of powdered forms. In reservoir DPIs, powder may be packaged in various forms, such as a loose cake or pressed shape in a reservoir. Examples of these types of DPIs include the Turbohaler™ inhaler (Astrazeneca, Wilmington, Del.) and the Clickhaler® inhaler (Innovata, Ruddington, Nottingham, UK). When a doctor blade or shutter (various forms of scraping devices) slides across the cake or shape, the powder is pushed into a stream of air whereby the patient can inhale the powder necessary for one therapeutic dose in a single large forced inspiration. These devices can contain upwards of 100 or more doses in a closed unit. Once the number of doses for which the unit has been designed has been dispensed, the devices must then be disposed. However, devices such as the Turbohaler require a very large single inspiratory breath (over 1000 mLs) in order for the dosage to be properly delivered to the lungs. A number of adults and many children cannot manage such a large inspiratory volume.

In comparison to reservoir DPIs, in “unit dose” DPIs, single or multiple doses of the powder are not preloaded. Rather, individual doses are inserted into the inhaler in the form of separate packages such as blisters, gelcaps, tabules, or other preformed vessels that have to be pierced, crushed, or otherwise unsealed to release the powder into a flow path for subsequent inhalation, again in a single large forced inhalation. Typically, all of the drug powder in one preformed vessel is dispensed at a time from the inhaler, though some designs or dosing regimens might call for additional drug powder preformed vessels to be inserted into the inhaler for immediate subsequent delivery in order to obtain the prescribed dose. Typical of these are the Diskus® inhaler (Glaxo, Greenford, Middlesex, UK), EasyHaler® (Orion, Expoo, FI), Novalizer®, Aerolizer® (Novartis, Basel, SZ), HandiHaler® (Boeringher-Ingleheim, Ingelheim, Del.), and UltraHaler® (Aventis).

In another existing configuration, once the package is pierced, pneumatic, mechanical or electrical agitators disperse all the powder from the package(s) and form an aerosol which keeps the drug powder suspended for a sufficient period of time to be inhaled by the patient in a single large forced inspiration. Examples of this are the Exubera® inhaler (Nektar, San Carlos, Calif.), Qdose inhaler (Microdose, Monmouth Junction, N.J.), and Spiros® inhaler (Dura, San Diego, Calif.).

Typical DPIs are designed and optimized to deliver as much of a single therapeutic dose as possible out of the inhaler into the patient's breath and into the lungs, in one single, large, forced inhalation in order to maximize delivery performance.

MDI and DPI systems generally only work if the patient is capable of coordinating a forced inhalation with each discharge of the aerosol upon a manual or breath actuation of the aerosol generator. They are intended to deliver a single dose for each actuation. For most therapies a maximum of 2 actuations is required for QD, BID or PRN.

There are many problems with these inhalers, resulting in wide variation in dosing consistency. In manually actuated MDIs and DPIs, patients frequently inhale too early or too late to effectively inspire the aerosol. Although a number of breath-actuated inhalers have been designed to address this problem, most of these devices cause discharge at the very onset of the patient's inspiratory effort. Depending on the pulmonary condition being treated and its location, it may often be more desirable for the active agent to be discharged near the peak of inhalation or later in the inhalation rather than the beginning. Further, it may be desirable to be able to selectively vary the point in the inhalation pattern at which the active agent is discharged in order to tailor the drug delivery to the condition being treated.

Another problem with MDIs and DPIs relates to the inhalation flow rate and inhalation volume, typically a forced inhalation, that is required to deliver a therapeutic dose to the respiratory tract. Typically, an inhalation flow rate greater than 30 liters per minute and an inhalation volume of at least one liter are required. Children, elderly or infirm individuals often cannot meet these inhalation requirements.

Some recent DPIs (e.g. Exubera®, made by Pfizer) are designed with an aerosol chamber. Typically one inhalation is required to draw down a standing cloud of powdered medicament. In such devices, a single aerosol cloud is generated after loading a powder-containing blister(s) into a large volume chamber. They require the patient to take one single deep inhalation (greater than 1 liter) to clear the entire aerosol cloud from the chamber. Because these devices do not regenerate the aerosol cloud with each breath, additional amounts of medicament need to be replenished by repeating the aforementioned steps with a new blister(s) of powder each time that additional powdered active agent needs to be dispensed.

Aqueous nebulizers deliver a streaming mist comprised of aqueous droplets containing drug, generated either on demand with each inhalation or continuously. The aerosol generator produces the mist from a small reservoir containing from 0.5 to 60 milliliters of a solution or suspension of the drug. The output flow rate of the aerosol mist is approximately matched to a lower flow rate, typically less than 6 liters per minute, allowing repetitive, non-forced inhalations, and continues until the liquid reservoir is emptied. Therefore the patient can administer the drug with repetitive, non-forced inhalations over a time period from approximately 1 minute to up to an hour, depending on the therapy. The concentration of the drug in the aerosol is relatively constant over the entire nebulization; therefore the patient inhales a consistent dose of the drug in every breath. The patient doesn't have to coordinate their inhalation maneuver with the actuation of the device. Therefore, nebulizers overcome the problems encountered by children, the elderly or infirm with MDIs and DPIs. Nebulizers allow the patient to effectively inhale the aerosol drug mist with repetitive non-forced breathing and eliminate the high-flow-rate forced inhalations required of other delivery techniques.

However traditional nebulizers also have the disadvantages of requiring external bulky air compressors attached to an electrical source to generate and deliver the drug-containing aerosol. The nebulization chamber must be cleaned frequently to function properly and to prevent the accumulation of microbial contamination. In addition, typical nebulizers require at least 5-10 minutes to deliver a full dose of the medicament, which becomes a time consuming and boring task that lowers compliance with the therapy. Recently “next generation’ aqueous nebulizers have been developed such as the eFlow (PARI), Aerodose (Aerogen), AERx (Aradigm), INeb (Respironics) that generate a soft mist by use of vibrating mesh or forcing fluid through an array of small holes. These devices are very efficient and reduce the nebulization time to less than 1 minute, allow non-forced inspirations, and are all breath actuated so they eliminate the need to manually coordinate the inhalation with device actuation. However, they have the disadvantages of all aqueous nebulizers in that they require significant effort to clean and maintain the device. Since they depend on forcing fluid through a fine mesh or array of holes, they are usually limited to low-concentration solutions or colloidal suspensions, thus limiting the payload that can be delivered. The mesh and holes are also easily damaged or clogged by the liquid formulations, resulting in a limited life span and increased maintenance costs. They are also usually an order of magnitude more expensive than traditional nebulizers, DPIs, or MDIs. Only a fraction of their cost is usually covered by healthcare payor reimbursement. Therefore their use and acceptance has been limited. Also delivery of active agents which are chemically or physically unstable in aqueous environments (hydrolabile) rules out the use of these active agents in nebulizers. Typically these agents must be protected from moisture by using them in DPIs or MDIs.

Thus, new inhalation devices and methods for delivering powdered active agents to the respiratory tract are desirable. In particular, breath-actuated inhalation devices with low cost, ease of use and maintenance, that operate with methods that deliver a consistent aliquot of a powdered active agent with each inhalation in a sequence of repetitive, non-forced or tidal breaths are desirable.

### SUMMARY

The dry powder inhalers of the present invention are breath-actuated, have no source of external mechanical, pneumatic or electrical energy, other than that provided by the patients breathing, and deliver the required amount of powdered active agent required for a particular administration over multiple inhalations utilizing repetitive non-forced breathing by the patient. The powdered active agent may be in stored in the dry powder inhaler in a dispensing unit in a form that permits multiple administrations. The dispensing unit may be replaceable by the patient. Alternatively, the powdered active agent is stored in replaceable preformed vessels which contain a define amount of powdered active agent. Typically one or two replaceable vessels would be used for a single administration. As used herein, administration refers to the delivery of a specified amount of powdered active agent over a relatively short period of time via a plurality of RNF inhalations.

The inhalers typically have a dose generating mechanism which creates an aerosol suspension of the active agent powder. The powder is either dispersed from a preformed vessel or in the alternative, created by ablation, abrading, abrasion or by some other mechanical means from the solid dosage form.

### BRIEF DESCRIPTION OF THE DRAWINGS

FIG. 1 is shows a schematic design of a first embodiment of the invention;

FIG. 2 shows a schematic design of a second embodiment of the invention

FIG. 3 shows a schematic design of a third embodiment of the invention.

### DETAILED DESCRIPTION

The inhalation devices (inhalers), kits, and methods of the present invention described herein relate to the delivery of powdered active agents to the lungs to treat asthma and other pulmonary conditions, including but not limited to allergic conditions and inflammatory conditions. The powdered actives are delivered over a series of Repetitive Non-Forced (RNF) breathing cycles on the part of the patient. RNF breathing, as used herein, is a series of inhalations and exhalations wherein the inspiratory volume and expiratory volume are approximately equal and both are less then or equal to 800 mLs and/or the flow rate is less than or equal to about 45 liters per minute and preferably less than or equal to about 30 liters per minute.

The inhalation devices of the present invention are adapted to deliver a therapeutic dose with RNF breathing cycles having inspiratory and expiratory volumes preferably in the range of about 50 milliliters to 800 milliliters, more preferably in the range of about 100 milliliters to about 600 milliliters, and most preferably in the range of about 150 milliliters to about 500 milliliters.

The inhalation devices of the present invention are adapted to deliver a therapeutic dose with RNF breathing cycles occurring preferably in the range of about 10 to about 40 breaths per min and most preferably in the range of about 12 to about 20 breaths per minute.

The inhalation devices of the present invention are adapted to deliver a therapeutically effective dose of a powdered active agent with a pressure drop across the device of preferably less than 40 millibar and most preferably less than 20 millibar.

The inhalation devices of the present invention are adapted to deliver a therapeutically effective dose of a powdered active agent when said RNF breathing has a flow rate of preferably about 5 to about 60 liters per minute, more preferably from about 10 to about 40 liters per minute and most preferably from about 15 to about 30 liters per minute.

Specifically, the inhalation devices are configured to release a predetermined amount of the active agent as fine aerosol with particles ranging in diameter from about 1 to about 10 median mass aerodynamic diameters (MMAD) and preferably from about 1 to about 5 MMAD. The aerosol is generated by dispersing a fraction of the initial unit dose of powder into a flow path of inhaled air, with each inhalation, thus delivering reproducible doses of the active agent over a series of RNF breathing cycles. In contrast to many commercially available breath-actuated inhalers, the inhalers described herein may provide an active ingredient powder suspension and deliver predetermined amount of active agent independent of the peak inspiratory flow rate generated, and specifically designed to accommodate RNF breathing cycles. They release the unit or metered reservoir dose in a series of small aliquots of the dose, actuated automatically with each of a series of inhalations, rather than in a single delivery occurring with a single forced inhalation as with existing DPIs and MDIs.

Further the dry powder inhalers of the present invention are designed to be Breath Actuated, which in the context of the embodiments of the present invention, means that the air flow caused by the patient's breath is the source of the energy used by the dose generating unit to form an aerosol of the dry powdered active agent. This can include one or more of the following steps: a) generating particles from a solid dosage form, b) dispersing the powder from a capsule, tabule, nebule or other preformed vessel and c) forming the aerosol suspension of the dry powder.

In addition to asthma, the dry powder nebulizer inhalation devices, kits, and methods described here may be used to treat pulmonary conditions that are selected from the group consisting of, without limitation, chronic obstructive pulmonary disease (COPD), including emphysema; cystic fibrosis; respiratory tract infections such as laryngitis, tracheitis, bronchitis, bronchiolitis, and those associated with Respiratory Synctial Virus, Cystic Fibrosis, and community and nosicomial pneumonia; neoplasms of the large and small airways; nasopharynx tumors; respiratory distress syndrome; and allergic or inflammatory conditions affecting the eyes, ears, or nose. As used herein, the terms “treat” or “treating” refer to the resolution of a pulmonary condition or the prevention or amelioration of a sign or symptom of a pulmonary condition, including signs or symptoms that are considered sequelae of the condition. For example, the dry powder nebulizers can be designed for the pediatric and elderly populations so that one or more doses of the powdered active agent are delivered according to the typical RNF breathing patterns of that population.

### I. Inhalation Devices

The inhalation devices may be of various designs, so long as they are capable of generating a powder suspension of an active agent upon breath-actuation, and delivering multiple (two or more) aliquots from the stored form of the powdered active agent over a series of RNF breathing cycles. The inhalation devices generally include a housing having a proximal end and a body portion. A mouthpiece will typically be positioned at the proximal end. One or more air inlets that allow air to flow through the housing are provided in the body portion. Examples of such housings are described in U.S. Pat. Nos. 6,418,926; 7,025,057; and 7,281,539. Unlike other commercially available breath-actuated devices for the pediatric population, it is not necessary to provide the inhalation devices described herein with a spacer to coordinate inhalation with release of the powder, or a chamber for containing the generated powder suspension until it has been completely inhaled.

### Examples of Modifications to Current Commercial Devices

There are several commercial and developmental dry powder devices that have been designed to be used under single forced inspiratory breath conditions. They use capsules or tabules filled with the drug which are punctured or have apertures punched in them once they are placed in the actuator/inhaler. These include the SpinHaler® from Fisons, Handihaler® from Boehringer Ingelheim, the Rotahaler® from GlaxoSmithKline, the Aerolizer® from Novartis, and the Orbital™ device described in U.S. Pat. No. 6,418,926 and U.S. Pat. No. 5,787,881. It serves to clarify the invention to describe how these devices would need to be modified to convert them to embody the inventive aspects of the present application and to be usable with a RNF breathing pattern.

Rotahaler®: In this device a single gel capsule filled with powdered active agent is placed into the well of the device. The capsule is sheared by rotating the upper cylinder to crush the capsule with a protruding wall. This opens the entire capsule by popping off the top to release the powder into the aerosolization chamber, whereupon it is inhaled in a single forced inspiratory breath of at least 30 liter per minute (LPM). To implement this embodiment of the invention, the Rotahaler would be modified to include a needle or multiple needles or a small doctor blade on the shearing wall protrusion of the device, such that when the shearing wall is actuated by turning the upper cylinder, only a small hole(s) would be punched into or a slice would removed from the capsule. The capsule contents would not be completely decanted into the chamber all at once because of the smaller openings that were formed. Thus, with each tidal breath only a small amount of the powder from within the capsule would be dispersed into the aerosolization chamber. Multiple breaths with flow rates lower than 30 LPM would be required to empty the capsule. A small increment of the powder would be released into each breath, with nominally at least 5 to 10 breaths required to empty the capsule.

Spinhaler®: In this device a single gel capsule is placed into the well of a spinning propeller mechanism that is in the flow path of the inspiratory breath. Upon closing the device large hole(s) are punched into each of the domed ends of the gel capsule. The inspiratory breath strikes the propeller which causes the gel capsule to spin. With a single forced inspiratory breath—usually greater than 30 LPM and optimally more than 45 LPM—the propeller spins and air passes through the gel capsule. The combined airflow and centrifugal force of the spinning capsule, driven by the rotation of the propeller, discharges all the drug powder from the capsule into the flow path in a single breath. To adapt this device to embody the inventive aspects of the present invention and thus deliver the desired quantity of powder active agent to a patient who is breathing with a RNF breathing pattern, a much smaller hole would be punched in each end of the gel capsule, for example with a 20 gauge or smaller needle. Alternatively only a single hole could be punched in the capsule in the distal end. In another variation, the propeller diameter would be decreased to allow it to spin under the influence of very low flow rates, but with a lower rotational velocity. Thus only a portion of the drug is discharged with each low-flow-rate RNF inhalation. A small increment of the powder would be released into each breath, with nominally at least 5 to 10 breaths required to empty the capsule.

HandiHaler® from Spiriva: In this device, a gel capsule in inserted into a well in the flow path of the inspiratory airflow. The device is then closed, which traps the capsule and a button is pushed to drive two large gauge (greater than 20 gauge) needles into the cylindrical walls of the capsule. One hole is placed proximally and one is placed distally along the flow path. Then the user inhales through the device with a flow rate of at least 30 LPM. The inspiratory airflow flows through and around the capsule causing it to shake and vibrate in the well. This combined airflow and the shaking causes the discharge of the entire contents from the capsule in a single forced inspiratory breath. To adapt the HandiHaler® to an RNF breathing pattern, a much smaller hole would need to be punched in the ends of the gel capsule, for example with a 20 gauge or smaller needle. Alternatively only a single hole could be punched in the capsule in the distal end. Additionally a vortexing flow generator can be installed at the air inlet below the capsule to impart a twisting flow on the capsule which will induce it to spin at very low flow rates. Thus only a portion of the drug is discharged with each low flow rate RNF inhalation. A small increment of the powder would be released into each breath, with nominally at least 5 to 10 breaths required to empty the capsule.

Foradil® Aerolizer: In this device a gel capsule is loaded into a reservoir, the device is closed and upon pressing buttons on the base of the device the capsule is punctured in 8 places by large gauge needles. Upon inhaling through the mouthpiece, the capsule is lifted up into an aerosolization reservoir where is spins rapidly and the drug powder is dispersed from the punctures under the influence of centrifugal force. This device requires flow rates in excess of 45 LPM and preferably greater than 60 LPM to operate optimally. To convert this device to an embodiment of the present invention which could be used with an RNF breathing pattern, the capsule would be punctured with needles that are smaller than 20 gauge and with a single hole in one end or a single hole in both ends. The aerosolization chamber size would be reduced to an effective radius no greater than 125% of the capsule length. This will allow a low inspirational flow to spin the capsule. Thus only a portion of the drug is discharged with each low-flow-rate RNF Inhalation. A small increment of the powder would be released into each breath, with nominally at least 5 to 10 breaths required to empty the capsule.

Orbital (Brin-Tech International, LTD.): This device operates in a manner similar to the Aerolizer with two notable exceptions. Instead of a capsule it uses a molded or stamped cylindrical shaped “tabule” with small apertures preformed on the side walls of the tabule. The inspiratory air flow induces the tabule to both orbit and spin around the inner wall of a circular or oval aerosolization chamber and while orbiting, it spins on its axis causing drug to be dispersed from the apertures (See FIG. 7, U.S. Pat. No. 6,418,926). Under normal usage the device requires flow rates greater than 45 LPM which causes the tabule to orbit the chamber from 20 to several hundred times per minute At the same time, the tabule will spin at a rate equal to the orbit rate times the ratio of the chamber radius to the tabule radius. This high orbit and spin rate contribute greatly to the disaggregation and dispersion of the drug. At lower flow rates, the tabule will orbit only a few times per minute and this results in much of the drug being deposited in the chamber, but not undergoing disaggregation, therefore remaining unaerasolizable. To modify this device to embody the present invention and thus enable it to be used with RNF breathing patterns, three key modifications must occur: the radius of the chamber must be reduced to below 1.0 cm, the ratio of chamber radius to tabule radius must be greater than 4:1 and the tabule will have only 1 or two apertures with slit widths less than 1 mm. These modifications allow the tabule to spin and orbit many times per minute even at low flow rates, such as below 30 LPM. At these low flow rates only a small portion of the drug is released with each RNF inhalation. A small increment of the powder would be released into each breath, with nominally at least 5 to 10 breaths required to empty the capsule.

The inhalation devices of the present invention further include a dose generating mechanism that is configured to provide an appropriate amount of the active agent in powdered form so that a predetermined amount is inhaled during RNF breathing. The devices are generally designed so that the air flow caused by the patients breathing actively powers the dose generating mechanism to generate a powder suspension of active agent in the flow path of air traveling from the one or more air inlets through the mouthpiece.

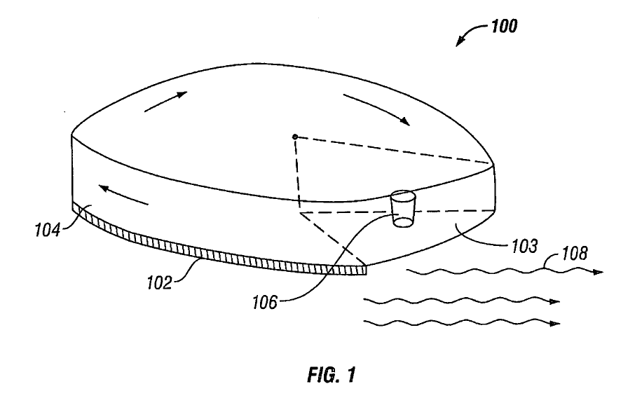

In one embodiment, is shown in FIG. 1. In this embodiment the Dose Generating Mechanism (100) includes a Fixed Base (102) having a Window (103) and a Rotating Tray (104). A Dosage Form (106) of active agent sits on the Rotating Tray (104), and is positioned on an Aperture (not shown) in the Tray (104). The dimensions of the Aperture will typically be sized to allow Dosage Form (106) to pass through the Tray (104) and into the Stream of Air (108) flowing towards the mouthpiece when the Tray (104) is rotated and Window (103) and the aperture are aligned. The amount of dosage form released can be controlled by adjusting how often the Window (103) and Aperture are aligned and how long they stay aligned. The Dosage Form (106) will usually disperse when contacted by the Stream of Air (108), but other disbursement or suspension generating mechanisms known in the art may also be employed. Although a shaped dosage form is depicted in FIG. 1, the active agent may also be contained on the Tray (104) as a powder. The particles pass through the Window (103) in the Fixed Base (102) when the aperture and Window (103) are aligned. Optionally, the aperture may be fitted with mesh-like cover to further control the amount of powder that is dispersed each time the Window (103) and the aperture are aligned. The number and/or dimensions of the Aperture(s) or Window (103) are additional means that may be adjusted to vary the amount of active agent delivered.

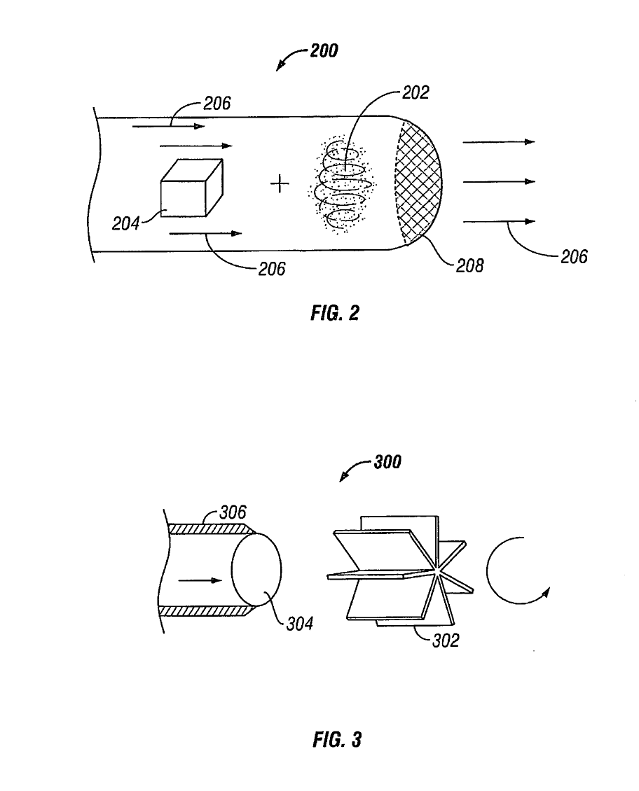

In another embodiment, as shown in FIG. 2, the Dose Generating Mechanism (200) employs Ablative Particles (202) to form an aerosol bolus of active agent particles. The active agent in this instance is provided as a Solid Dosage Form (204). The Ablative Particles (202) are suspended by air flow caused by the patient's breathing. Upon exposure to a surface of the Solid Dosage Form (204) the suspension of Ablative Particles (202) grinds off a predetermined amount of the Dosage Form (204). Prior to the Stream of Air (206) moving outside of the Dose Generating Mechanism (200) and traveling towards the mouthpiece, a Filter (208) may be used to remove the Ablative Particles (202) from the powder suspension.

In yet a further embodiment, as illustrated in FIG. 3, the Dose Generating Mechanism (300) includes a Blade (302). The Blade (302) is constructed so that upon rotation and contact with a Solid Dosage Form (304), a portion of the Solid Dosage Form (304) is scraped off to form a dry powder. Rotation of the Blade (302) is caused by the patient's RNF breathing. As shown in FIG. 3, Solid Dosage Form (304) is stored in Dispensing Unit (306), which has a tubular structure. Contact between the Blade (302) and Solid Dosage Form (304) may be achieved by incrementally advancing the Dosage Form (304) from the Dispensing Unit (306). In other instances, contact may be made by pressing the Blade (302) against the Solid Dosage Form (304). A spring (not shown) may be used in this instance to aid the Blade (302) in applying pressure against the Solid Dosage Form (304). Although the Blade (302) in FIG. 3 is star-shaped, other blade configurations may be implemented.

The dry powder inhalation devices of the present invention may be configured to provide a predetermined amount of active agent, based on such parameters as time spent breathing from the inhalation device, number of inhalations, flow rate during RNF breathing, and/or the minute ventilation typically observed in a particular population. For example, these dry powder inhalation devices may be designed to deliver a predetermined amount of powdered active agent during at least about 30 seconds of RNF breathing or with about 12 to 40 breaths per minute. With respect to minute ventilation, the inhalation devices may be constructed to provide a predetermined amount of active agent at minute ventilations between about 1.0 liters in one minute to about 30 liters in one minute. As previously mentioned, delivery of the predetermined amount of powdered active agent may be made to be independent of peak inspiratory flow rate, if desired.

Furthermore, the inhalation devices of the present invention may be fitted with an inhalation-only valve, or other back-flow prevention elements, in order to prevent exhaled, moist, breath from entering the device and causing powder agglomeration or contamination on the internal device surfaces. Alternatively, the patient can instructed to exhale with the device moved away from the patient's mouth in order to prevent the exhaled moist air from being directed back into the inhaler.

Any active agent may be included in the inhaler devices described herein so long as they are suitable to treat systemic or local conditions, including, but not limited to, asthma or other pulmonary conditions, and the allergic or inflammatory conditions previously mentioned, and are capable of being formulated in powdered form. The active agents are typically formulated as solid particles or solid shapes of various geometry and/or porosities.

In one variation, the active agent is budesonide. Reference to budesonide includes, but is not limited to, any form of budesonide that may be used to treat asthma or COPD, including, but not limited to, pharmaceutically acceptable derivatives, analogues, enantiomer forms, stereoisomers, anhydrides, acid addition salts, base salts, and solvates.

Other active agents that may be used include, but are not limited to, anti-infective agents, anti-inflammatory agents, and chemotherapeutic agents. Anti-infective agents generally include antibacterial agents, antifungal agents, antiparasitic agents, and antiviral agents. Anti-inflammatory agents generally include steroidal and nonsteroidal anti-inflammatory agents.

Examples of antibacterial agents that may be suitable for use with the described inhalers, kits, and methods include, but are not limited to, aminoglycosides, amphenicols, ansamycins, β-lactams, lincosamides, macrolides, nitrofurans, quinolones, sulfonamides, sulfones, tetracyclines, vancomycin, compounds and complexes of metals such as gallium, indium, zinc, silver, and copper, and any derivatives, analogues, or combinations of any of the aforementioned, thereof.

Examples of antifungal agents suitable for use with the described inhalers, kits, and methods include, but are not limited to, allylamines, imidazoles, polyenes, thiocarbamates, triazoles, and any of their derivatives. In one variation, imidazoles are the preferred antifungal agents. Antiparasitic agents that may be employed include such agents as atovaquone, clindamycin, dapsone, iodoquinol, metronidazole, pentamidine, primaquine, pyrimethamine, sulfadiazine, trimethoprim/sulfamethoxazole, trimetrexate, and any of their derivatives, analogues, or combinations thereof.

Examples of antiviral agents that may be used include, but are not limited to, acyclovir, famciclovir, valacyclovir, edoxudine, ganciclovir, foscamet, cidovir (vistide), vitrasert, formivirsen, HPMPA (9-(3-hydroxy-2-phosphonomethoxypropyl)adenine), PMEA (9-(2-phosphono-methoxyethyl)adenine), HPMPG (9-(3-Hydroxy-2-(Phosphonomet-1-hoxy)propyl)guanine), PMEG (9-[2-(phosphonomethoxy)ethyl]guanine), HPMPC (1-(2-phosphonomethoxy-3-hydroxypropyl)-cytosine), ribavirin, EICAR (5-ethynyl-1-beta-D-ribofuranosylimidazole-4-carboxamine), pyrazofurin (3-[beta-D-ribofuranosyl]-4-hydroxypyrazole-5-carboxamine), 3-Deazaguanine, GR-92938X (1-beta-D-ribofuranosylpyrazole-3,4-dicarboxami-1-de), LY253963 (1,3,4-thiadiazol-2-yl-cyanamide), RD3-0028 (1,4-dihydro-2,3-Benzodithiin), CL387626 (4,4′-bis[4,6-d][3-aminophenyl-N—,N-bis(2-carbamoylethyl)-sulfonilimino]-1,3,5-triazin-2-ylamino-biphenyl-2-,2′-disulfonic acid disodium salt), BABIM (Bis[5-Amidino-2-benzimidazoly-1]-methane), NIH351, gallium, indium and silver compounds and complexes and any of their derivatives, analogues, or combinations thereof.

Typically, a steroidal anti-inflammatory agent, e.g., a corticosteroid, is formulated for use with the inhaler devices, kits, and methods described herein. Exemplary steroidal anti-inflammatory agents include 21-acetoxypregnenolone, alclometasone, algestone, amcinonide, beclomethasone, betamethasone, chloroprednisone, ciclesonide, clobetasol, clobetasone, clocortolone, cloprednol, corticosterone, cortisone, cortivazol, deflazacort, desciclesonide, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, any of their derivatives, analogues, and combinations thereof.

If a nonsteroidal anti-inflammatory agent is used, suitable agents include, but are not limited to, COX inhibitors (COX-1 or COX nonspecific inhibitors) (e.g., salicylic acid derivatives, aspirin, sodium salicylate, choline magnesium trisalicylate, salsalate, diflunisal, sulfasalazine and olsalazine; para-aminophenol derivatives such as acetaminophen; indole and indene acetic acids such as indomethacin and sulindac; heteroaryl acetic acids such as tolmetin, dicofenac and ketorolac; arylpropionic acids such as ibuprofen, naproxen, flurbiprofen, ketoprofen, fenoprofen and oxaprozin; anthranilic acids (fenamates) such as mefenamic acid and meloxicam; enolic acids such as the oxicams (piroxicam, meloxicam) and alkanones such as nabumetone) and selective COX-2 inhibitors (e.g., diaryl-substituted furanones such as rofecoxib; diaryl-substituted pyrazoles such as celecoxib; indole acetic acids such as etodolac and sulfonanilides such as nimesulide).

Suitable chemotherapeutic/antineoplastic agents that may be used include, but are not limited to antitumor agents (e.g., cancer chemotherapeutic agents, biological response modifiers, vascularization inhibitors, hormone receptor blockers, cryotherapeutic agents or other agents that destroy or inhibit neoplasia or tumorigenesis) such as alkylating agents or other agents which directly kill cancer cells by attacking their DNA (e.g., cyclophosphamide, isophosphamide), nitrosoureas or other agents which kill cancer cells by inhibiting changes necessary for cellular DNA repair (e.g., carmustine (BCNU) and lomustine (CCNU)), antimetabolites and other agents that block cancer cell growth by interfering with certain cell functions, usually DNA synthesis (e.g., 6-mercaptopurine and 5-fluorouracil (5FU), antitumor antibiotics and other compounds that act by binding or intercalating DNA and preventing RNA synthesis (e.g., doxorubicin, daunorubicin, epirubicin, idarubicin, mitomycin-C and bleomycin) plant (vinca) alkaloids and other anti-tumor agents derived from plants (e.g., vincristine and vinblastine), steroid hormones, hormone inhibitors, hormone receptor antagonists and other agents which affect the growth of hormone-responsive cancers (e.g., tamoxifen, herceptin, aromatase ingibitors such as aminoglutethamide and formestane, trriazole inhibitors such as letrozole and anastrazole, steroidal inhibitors such as exemestane), antiangiogenic proteins, small molecules, gene therapies and/or other agents that inhibit angiogenesis or vascularization of tumors (e.g., meth-1, meth-2, thalidomide), bevacizumab (Avastin), squalamine, endostatin, angiostatin, Angiozyme, AE-941 (Neovastat), CC-5013 (Revimid), medi-522 (Vitaxin), 2-methoxyestradiol (2ME2, Panzem), carboxyamidotriazole (CAI), combretastatin A4 prodrug (CA4P), SU6668, SU11248, BMS-275291, COL-3, EMD 121974, IMC-1C11, IM862, TNP-470, celecoxib (Celebrex), rofecoxib (Vioxx), interferon alpha, interleukin-12 (IL-12), biological response modifiers (e.g., interferon, bacillus calmette-guerin (BCG), monoclonal antibodies, interleukin 2, granulocyte colony stimulating factor (GCSF), etc.), PGDF receptor antagonists, herceptin, asparaginase, busulphan, carboplatin, cisplatin, carmustine, cchlorambucil, cytarabine, dacarbazine, etoposide, flucarbazine, fluorouracil, gemcitabine, hydroxyurea, ifosphamide, irinotecan, lomustine, melphalan, mercaptopurine, methotrexate, thioguanine, thiotepa, tomudex, topotecan, treosulfan, vinblastine, vincristine, mitoazitrone, oxaliplatin, procarbazine, streptocin, taxol or paclitaxel, taxotere, analogues/congeners, gallium compounds and complexes, derivatives of such compounds, and combinations thereof.

The formulations that may be used with the inhaler devices described herein may also include excipients and/or additives. Suitable excipients and/or additives that may be employed are found in U.S. Publication No. 2007/0178051. For example, the formulations may include one or more antioxidants or chelating agents. Chelating agents include, but are not limited to, cyclodextrins, cromoglycates, xanthates including caffeine, pegylation agents, crown ethers, ethylenediaminetetraacetic acid (EDTA) or a salt thereof, such as the disodium salt, citric acid, nitrilotriacetic acid and the salts thereof. Antioxidants include, but are not limited to, vitamins, provitamins, ascorbic acid, vitamin E, or salts or esters thereof.

Other excipients that may be used, include, but are not limited to, one or more pH modifiers, binding agents, filling agents, lubricating agents, suspending agents, sweeteners, flavoring agents, preservatives, wetting agents, and disintegrants.

Examples of suitable filling agents are lactose monohydrate, lactose anhydrous, and various starches. Examples of binding agents are various celluloses and cross-linked polyvinylpyrrolidone, microcrystalline cellulose, such as Avicel® PH101 and Avicel® PH102, microcrystalline cellulose, and silicifized microcrystalline cellulose (SMCC).

Suitable lubricants, including agents that act on the flowability of the powder to be compressed, are colloidal silicon dioxide, such as Aerosil® 200; talc, stearic acid, magnesium stearate, calcium stearate, and silica gel.

Examples of suitable sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of suitable preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of suitable diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Components of the inhalation device may be manufactured from materials conventionally utilized in inhalation drug delivery devices. Exemplary materials include plastics materials such as polyesters, polysulfones, polystyrenics, acetals, cellulosics, polyamides, polycarbonate, polyolefins such as polypropylene or polyethylene, and the like. Other materials such as metals, including aluminum and stainless steel, may also be used. Combinations of materials may also be employed so that the most suitable material is used for each component. For example, the dispensing unit, which stores the active agent, may be formed from a material that has the requisite impermeability to moisture. In one variation, the material used to make the dispensing unit is a metal such as aluminum, stainless steel, or alloys thereof. In other variations, plastics materials may be used to form the dispensing unit. Suitable plastics include without limitation, high density polyethylene, polycarbonate, polyvinylchloride, polyethylene terephthalate, and polypropylene.

### II. Methods

Methods for delivering a powdered active agent over a series of RNF inhalations are also described. In general, the method employs providing an inhalation device having a dispensing unit and dose generating mechanism as previously described. The patient or user then raises the device to his mouth and inhales through the mouthpiece and engages in RNF breathing. The inspiratory air flow provides the energy necessary to activate the dose generating mechanism which then generates the active agent in powdered form. The powdered active agent can either be generated and then introduced into the steam of air or it is generated directly within the stream of air which is drawn into the housing through the air inlets. RNF breathing is maintained, and a predetermined amount of the active agent is delivered over a plurality of RNF inhalations according to various target parameters programmed in the device.

Methods for treating asthma and other pulmonary conditions are also described. In general, the method involves providing a powdered formulation containing an active agent for treating the pulmonary condition in one or more unit dose forms and administering the formulation over a series of inhalations while the patient maintains a tidal breathing pattern.

The dry powder of the active agent may be delivered to any structure or tissue within the respiratory system. For example, the active agent may be delivered to the larynx, trachea, bronchi, bronchioles, alveoli, or any combination thereof. In one variation, one or more predetermined amounts of budesonide are provided to the respiratory system by the inhalers described herein during RNF breathing. Methods for treating allergic and inflammatory conditions involve the same or similar steps as those described above.

### III. Kits

The inhalation devices of the present invention described herein may be included in kits for delivering powdered active agents to the respiratory system during RNF breathing. The kits may include one or more dispensing units for use with the inhaler. The dispensing units may include the same active agent or different active agents. Optionally the kits may include preformed vessels containing predetermined amounts of one or more active agents which may be inserted in the dose generating units.

The kit may also include instructions that the patient is only to use the inhaler with RNF breathing and not to use forced inhalations. The instructions may also include how to operate the inhaler during RNF breathing, how to remove and replace the dispensing unit, how to insert and remove the preformed vessels, and how long RNF breathing should be performed in order to fully delivery the content of the preformed vessels.

While the invention has been described in conjunction with the specific embodiments described herein, it is to be understood that the foregoing description as well as the examples are intended to illustrate and not limit the scope of the invention. Other aspects, advantages and modifications within the scope of the invention will be apparent to those skilled in the art to which the invention pertains.

1. A dry powder nebulizer for the pulmonary delivery of an active agent powder to a patient comprising:
a) a housing having a first end and a body portion;
b) a mouthpiece disposed at said first end of said housing;
c) a dose generator disposed within the body portion of said housing;
said dose generator further comprising a dosage form of the active agent; and
d) one or more air inlets in said body portion in fluid communication
with the mouthpiece and said dispensing unit;
e) wherein said dry powder nebulizer is configured to provide a therapeutically effective dose of said active agent in powdered form by dispensing and aerosolizing said active agent into a stream of air flowing from the one or more air inlets through the mouthpiece and to the patient over a plurality of RNF breathing cycles.

2. A dry powder nebulizer as described in claim 1 wherein said dry powder nebulizer is breath-actuated.

3. The dry powder nebulizer of claim 1 wherein said dosage form comprises the active agent as particles

4. The dry powder nebulizer of claim 1 wherein said dosage form is a shaped solid mass.

5. The dry powder nebulizer of claim 1 wherein the dose generator comprises a rotating tray having an aperture and a fixed base member having a window, wherein alignment of the aperture and window allows the active agent to be aerosolized and dispensed into the air flowing from the one or more air inlets through the mouthpiece.

6. The dry powder nebulizer of claim 1 wherein the dose generator comprises a plurality of ablative particles adapted to grind a predetermined fraction of the dosage form of the active agent to form a powder which is aerosolized and dispensed into the air flowing from the one or more air inlets through the mouthpiece.

7. The dry powder nebulizer of claim 1 wherein the dose generating mechanism comprises a rotating blade which forms particles of the active agent from a shaped solid dosage form, wherein said particles are aerosolized and dispensed into the air flowing from the one or more air inlets through the mouthpiece.

8. The dry powder nebulizer as described in claim 1 wherein the active agent is selected from the group consisting of anti-infective agents, anti-inflammatory agents, chemotherapeutic agents, and combinations thereof.

9. The dry powder nebulizer as described in claim 8 wherein the active agent comprises an anti-inflammatory agent.

10. The dry powder nebulizer as described in claim 9 wherein the anti-inflammatory agent comprises a steroidal or nonsteroidal anti-inflammatory agent.

11. The dry powder nebulizer as described in claim 10 wherein the steroidal anti-inflammatory agent comprises budesonide.

12. The dry power nebulizer as described in claim 1 which is adapted to deliver said therapeutic dose when the volume of said RNF breath cycles are in the range about 50 to about 800 milliliters.

13. The dry power nebulizer as described in claim 1 which is adapted to deliver said therapeutic dose when the volume of said RNF breaths are in the range about 100 to about 600 milliliters.

14. The dry power nebulizer as described in claim 1 which is adapted to deliver said therapeutic dose when the volume of said RNF breaths are in the range about 150 to about 500 milliliters.

15. The dry power nebulizer as described in claim 1 which is adapted to deliver said therapeutic dose when said RNF breathing cycles occur at the rate of about 10 to about 40 breaths per minute.

16. The dry power nebulizer as described in claim 1 which is adapted to deliver said therapeutic dose when said RNF breathing cycles occur at the rate of about 12 to about 20 breaths per minute.

17. The dry power nebulizer as described in claim 2 wherein said dosage form is removably attached to said dry power nebulizer.

18. The dry powder nebulizer as described in claim 2 further comprising a plurality of dosage forms, wherein said dosage forms are removably attached to said dry power nebulizer.

19. The dry powder nebulizer as described in claim 2 wherein the pressure drop across said dry powder nebulizer during use is less than about 40 millibars.

20. The dry powder nebulizer as described in claim 2 wherein the pressure drop across said dry powder nebulizer during use is less than about 20 millibars.

21. The dry powder nebulizer as described in claim 2 wherein said nebulizer is adapted to deliver a therapeutically effective dose of said active agent when the flow rate of said RNF breathing cycles is in the range of about 5 to about 60 liters per minute.

22. The dry powder nebulizer as described in claim 2 wherein said nebulizer is adapted to deliver a therapeutically effective dose of said active agent when the flow rate of said RNF breathing cycles is in the range of about 10 to about 40 liters per minute.

23. The dry powder nebulizer as described in claim 2 wherein said nebulizer is adapted to deliver a therapeutically effective dose of said active agent when the flow rate of said RNF breathing cycles is in the range of about 15 to about 30 liters per minute.

24. The dry powder nebulizer as described in claim 2 wherein said nebulizer is adapted to deliver a therapeutically effective dose of said active agent when pressure drop across said dry power nebulizer is less than about 40 millibars.

25. The dry powder nebulizer as described in claim 2 wherein said nebulizer is adapted to deliver a therapeutically effective dose of said active agent when pressure drop across said dry power nebulizer is less than about 20 millibars.

26. A method for delivering an active agent to a patient comprising:
providing the dry powder nebulizer of claim 1 and using said dry powder nebulizer to deliver a therapeutically effective dose of an active agent in powder form to the respiratory airway of a patient having a respiratory condition, during a plurality of RNF breathing cycles.

27. The method as described in claim 26 wherein said patient is suffering from a respiratory condition selected from the group comprising asthma and chronic obstructive pulmonary disease.
